(19) United States
(12) Patent Application Publication
(10) Pub. No.: US 2004/0131571 A1
(43) Pub. Date: Jul. 8, 2004

(54) Cosmetic product for skin care based on a three-element molecular complex

(76) Inventor: Jose Eisenberg, Monaco (MC)

Correspondence Address: YOUNG & THOMPSON, 745 SOUTH 23RD STREET 2ND FLOOR, ARLINGTON, VA, 22202

(21) Appl. No.: 10/475,353

(22) PCT Filed: Apr. 6, 2002
(86) PCT No.: PCT/EP02/03826

(30) Foreign Application Priority Data
Apr. 19, 2001 (MC) 2464

(51) Int. Cl. A61K 7/06; A61K 7/11

(52) U.S. Cl. 424/70.14; 424/85.1

(57) ABSTRACT

The invention concerns a cosmetic product for skin care based on a three-element molecular complex. It comprises a combination of three active principles: enzymes, cytolines, biostimulins. The enzymes produce a keratolytic effect on the skin; the cytolines act to stimulate the cell activity, including the cell renewal; the biostimulins act to promote oxygenation of the cells, which constitutes a vital energy source. The invention is applicable in cosmetology.

The invention has for its object a cosmetic product for treating the epidermis based on a complex molecular trio.

The state of the art can be defined by the following patents which describe the activity of cytokins.

The patent EP 0 826 367 discloses the action of an extract of bertholletia to stimulate the synthesis of collagen.

The invention relates to new uses for an extract of bertholletia in the cosmetic or pharmaceutical field, particularly dermatology and for the preparation of cell culture media.

It relates more particularly to the uses in the cosmetic or pharmaceutical field in which it is desired to stimulate the synthesis of collagen VII and/or to promote the incorporation of vitamin C in the skin cells, in particular in the fibroblasts.

It relates also to new cell culture media containing an extract of bertholletia.

The patent FR-A-2743817:

The present invention relates to a model of reconstructed skin, characterized by the fact that it comprises an epidermal equivalent on a support, said epidermal equivalent comprising at least keratinous sites and Langerhans cells and/or precursors of Langerhans cells as well as the process for producing said skin equivalent.

The invention also relates to an epidermal equivalent, characterized by the fact that it comprises at least keratinous sites and Langerhans cells and/or precursors of Langerhans cells and its process for preparation.

The cosmetic product according to the invention has for its object to use active principles which, in combination, reactivate the characteristics of the skin so that it remains flexible, toned and elastic.

The invention resides in the fact of combining three molecules, each having its own function, enzymes, cytokins and biostimulins.

The combination of these three functions provides a spectacular result, particularly visual, on the skin.

The enzymes have a keratolitic effect on the skin.

The cytokins have for their function to stimulate the cellular activity, namely cellular renewal.

The biostimulants have for their function to promote the oxygenation of the cells, a vital source of energy.

The synthesis of collagen and or elastin guaranteeing the toning and elasticity of the skin.

The enzymes are Bacillus Subtilis.

The cytokins are polypeptide extracts of Lactis Proteinum milk.

The biostimulants are extracts of fresh tissues of young beech shoots, Fagus Silvatica.

The active principles used in combination to interact are:

Enzymes with keratolitic effect. These biotechnological molecules have the role of digesting and eliminating the desquamant corneocytes.

They promote a perfect assimilation of the active principles and guarantee a better quality of the cellular mantle.

Cytokin molecules are the key to cellular auto-control. The cytokins are messengers of a peptidic nature emitted by certain cells (white globules, cells of the immune system). They act on the receptors of other cells to induce numerous vital reactions, among them cellular renewal.

The cytokins are extracted from milk. These are selected polypeptides (amino acid groups), bio-active, with a certain regulated and verifiable biological activity.

Biostimulants oxygenating the cells and promoting the protein synthesis. The biostimulants of fresh tissues of young beech shoots, whose composition is very near proteins of our cutaneous tissue, promote:

The oxygenation of the cells, a source of vital energy

The synthesis of collagen and elastin guaranteeing toning, elasticity.

The enzymes with keratolitic effect. By fermentation of a microorganism: Bacillus Subtilis, there is extracted an enzyme (a protease) whose capacity resides in the elimination of the desquamant corneocytes. The natural enzymatic action permits carrying out under mild conditions the cleaning of the skin and thus accelerating its renewal. It is staged over time, as a function of need. The dead kerotin of the corneocytes is digested, the cellular cohesion of the desquamant cells is broken, the skin can renew itself more rapidly.

The biostimulins are obtained from young fresh tissue of beech (Fagus Silvatica L). At a short and precise time of the year (time of the harvest), these tissues are concentrated in vegetable biostimulins.

The amino acids of these biostimulins are similar to the structural proteins of the cutaneous tissue (collagen). They are also rich in essential molecules (mineral salts, peptides) and amino acids (proline, tryptophane, alanine) which play a basic role in cellular metabolism.

The biostimulins used in the amount of 20% increase the consumption of oxygen by 71% (8830 oxygraphic units) and hence a very important oxygraphy.

The biostimulins are non-cytotoxic as to fibroblasts and keratinocytes, which keratinocytes benefit the most from the action of the product at the cutaneous level.

The biostimulins can have an action on the synthesis of essential proteins produced by keratinocytes such as Keratin, filaggrin, involucrin, transport proteins, immune proteins . . . .

An increase of 42% of the protein synthesis is effected by the addition of 0.4% of biostimulins.

The biostimulins do not modify the morphology of the cells with which they are in contact and do not give rise to any cellular proliferation.

Aging is a dynamic process which acts on all of the body at different levels.

In addition to the natural aging process, genetically programmed, there are external disturbances which give rise to a modification of the structural/functional relations in the skin: it is thus necessary to make a distinction between biological aging and accelerated (premature) aging.

The structural/functional relationships take place in the skin between the extracellular matrix and the cells.

The extracellular matrix is a highly organized and complex compartment, constituted by different types of collagen, elastin, proteoglycanes, laminine and fibronectine.

A cell must maintain constant molecular information on its extracellular compartment so as to adapt itself to the variations of its environment.

This information is transmitted via cellular receptors and the integrins (adhesive molecules) which permit coordinating the activity of the cell with that of the tissue to which it relates (functions).

A knowledge of the mechanisms of regulation and their control factors is widespread.

The general term of cytokin covers a certain number of definite polypeptides. These represent the information system for the cells both as to molecular signals and as to growth or inhibition factors, and they perform a definite task. These molecules interact with the cells thanks to specific receptors.

The cytokins control the good maintenance and re-establishment of homeostasy, functional equilibrium between the cells and their extracellular environment.

The cytokins are key molecules for cellular control. These components interact in a coordinated fashion so as to restore and maintain cutaneous integrity.

The cytokins, milk is itself a nearly complete food to maintain life, but milk is always more than one food source. It has been established that milk contains a vast assortment of biologically active polypeptides capable of stimulating and regulating, but also of inhibiting a certain number of cellular functions.

An increasing number of tests permit affirming that the peptides of milk have real physiological functions (to have a view of this question, cf. Britton and Kastin, Am. J. Med. Sci. 1991, 302(2), 124-132).

The role of certain of these peptides of milk has been established: they act as natural mediators of the cellular processes charged with maintaining the skin in a good condition (cf. Pittelkow, Advances in Dermatology vol. 7.55-81 (1991)).

Such polypeptides are part of the peptide growth factors and belong to the general class of cytokins.

It has been possible to isolate from milk most of the more important cytokins:

Epidermic Growth Factor (EGF) (Carpenter, 1980; Beardmore, Richards, 1983; Pétrides et al., 1985; Yagi et al., 1986)

Transformation Growth Factor α (TGF-α) (Sweibel et al., 1986); Okada et al., 1991)

Transformation Growth Factor β-1 and β-2 (TGF-β1, 2) (Cox, Burk, 1991; Jin et al., 1991)

Colonies Stimulation Factor (CSF) (Sinha, Yunis, 1983)

Pseudo-insulin Growth Factor I and II (IGF) (Suikkari, 1989; Nagashima et al., 1990; Donovan et al., 1991)

Interleukene-1 and 2 (IL-1, IL-2) (Söder, 1987; Britton et al., 1991)

Nerve Growth Factor (NGF) (Britton et al., 1991)

The cytokins present in milk are in a deactivated form. By a process of several steps it has been possible starting from milk to restore them to their biologically active form.

This complex also contains lactalbumin, lactose, lactoglobulin, lactoferrin. These components have their own value and function as cosmetic substances, but the essential function is above all to stabilize the activated cytokins.

1. Cosmetic product to reactivate the characteristics of the skin so that it will remain flexible, toned and elastic,
characterized by the fact,
that it comprises a combination of three active principles:
enzymes
cytokins
biostimulins

2. Cosmetic product according to claim 1, characterized by the fact,
that the enzymes have a keratolitic effect on the skin; the cytokins have the function of stimulating cellular activity, including cellular renewal; the biostimulins have the function of promoting oxygenation of the cells, a source of vital energy.

3. Cosmetic product according to any one of claims 1 to 2,
characterized by the fact,
that the enzymes are Bacillus Subtilis.

4. Cosmetic product according to any one of claims 1 to 2,
characterized by the fact,
that the cytokins are polypeptides extracted from Lactis Proteinum milk.

5. Cosmetic product according to any one of claims 1 to 2,
characterized by the fact,
that the biostimulins are extracts of young shoots of beech, Fagus Silvatica.
